(19) United States
(12) Patent Application Publication
(10) Pub. No.: US 2010/0203146 A1
(43) Pub. Date: Aug. 12, 2010

(54) INTERMITTENT DOSING STRATEGY FOR TREATING RHEUMATOID ARTHRITIS

(75) Inventor: Gary S. Jacob, New York, NY (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Callisto Pharmaceuticals, Inc., New York, NY (US)

(21) Appl. No.: 12/702,697

(22) Filed: Feb. 9, 2010

Related U.S. Application Data
(60) Provisional application No. 61/151,112, filed on Feb. 9, 2009.

(51) Int. Cl. A61K 9/14; A61K 31/40; A61K 31/4985; A61K 33/24; A61K 38/13; A61K 31/42; A61K 31/52; A61K 31/4402; A61K 31/7135; A61P 19/02

(52) U.S. Cl. 424/489; 514/409; 514/249; 424/649; 514/9; 514/378; 514/263.2; 514/353; 514/24

(57) ABSTRACT

The present invention provides methods of treating rheumatoid arthritis. The methods generally involve an intermittent dosing strategy for administering Atiprimod alone or in combination with a second therapeutic agent wherein the second therapeutic agent is selected from the group consisting of Methotrexate, gold compounds, antimalarials, cyclosporine A, leflunomide, azathioprine, sulfasalazine d-penicillamine, aurothioglucose, auranofin, or a TNF inhibitor.

### RELATED APPLICATION

This non-provisional patent application claims priority under 35 U.S.C. §119(e) to U.S. Provisional Patent Application Ser. Nos. 61/151,112, filed Feb. 9, 2009, which is incorporated herein.

### BACKGROUND

Rheumatoid arthritis (RA) is an autoimmune disease that causes inflammation of the joints, the tissue around the joints, as well as other organs in the body. RA is a progressive illness that can lead to long-term joint damage, chronic pain, loss of function and disability. RA most commonly affects the wrists, fingers, knees, feet, and ankle joints. Symptoms of RA include increased pain and stiffness in the morning, swollen joints, deformed and misshapen joints, intense fatigue, decreased energy, muscle aches, decreased appetite, weight loss, fever, depression, problems sleeping, anemia, bumps occurring under the skin (rheumatoid nodules), inflamed blood vessels, bleeding stomach ulcers, inflammation of the heart's sac (pericarditis), inflammation of the heart muscle (myocarditis), lung problems and eye problems.

The treatment of RA remains a major challenge. The major goals of therapy are to relieve pain, swelling, and fatigue, and to improve joint function, stop joint damage, and prevent disability and disease related morbidity. Current methods of treating the disease may involve a combination of two or more therapeutics compounds, including non-steroidal anti-inflammatory drugs, (“NSAIDs”, e.g., ibuprofen), COX-2 inhibitors (e.g., celecoxib (Celebrex)), low dose corticosteroids, disease-modifying anti-rheumatic drugs (“DMARDs”, e.g., Methotrexate, gold compounds, antimalarials, cyclosporine A, leflunomide, azathioprine, sulfasalazine d-penicillamine, aurothioglucose or auranofin), Tumor necrosis factor (“TNF”) inhibitors (e.g., etanercept (Enbrel), infliximab (Remicade), and adalimumab (Humira), interleukin-1 inhibitors (e.g., injectible anakinra (Kineret)), and other biologic response modifiers (“BRMs”).

Accordingly, while society has seen tremendous advances in the field of pharmaceuticals, there are, of course, drawbacks to the administration of any given pharmaceutical agent. Sometimes, the disadvantages, characterized as “side effects,” are so severe as to preclude administration of a particular agent at a therapeutically effective dose. For example, these drugs may be effective only in a subset of patients and their long term use is limited by side effects, some of which are severe.

In such a case, drug therapy is discontinued, and other pharmaceutical agents may be tried. Many agents in the same therapeutic class, however, display similar side effect profiles, meaning that patients either have to forego therapy or suffer from unpleasant side effects associated with a particular medication.

The present invention is directed to an intermittent dosing strategy for administering Atiprimod alone or in combination with a second therapeutic agent which directly or indirectly reduces the side effects associated with one or both of the agents administered. By “indirectly” reducing side effects is meant that a first pharmaceutical agent allows the second agent to be administered at a lower dose without compromising therapeutic efficacy, thus resulting dose-dependent unwanted effects. In particular, the long half-life of Atiprimod (˜60 days, in man) presents a challenge. This invention endeavors, on the basis of some unexpected findings, to turn the long half-life of atiprimod into an advantage by employing a novel, intermittent dosing strategy that has not been previously described.

While the invention is useful in conjunction with numerous pharmaceutical agents and therapeutic regimens, conditions of particular interest that may be treated according to the present methodology include rheumatoid arthritis.

Atiprimod (2-(3-Diethylaminopropyl)-8,8-dipropyl-2-azaspiro[4,5]decane) was originally developed by SmithKline Beecham as a treatment for rheumatoid arthritis (RA) and successfully completed three Phase 1 clinical trials(1) in RA patients. These trials included single-dose studies, one-month multiple-dose studies and an open-label(2) extension study in which patients were on the drug for as long as one year. The results of these studies showed the drug to be well-tolerated at the administered doses, with no observed dose-limiting toxicity.

The treatment of RA has undergone a dramatic change since the introduction of novel disease modifying antirheumatic drugs (DMARDs). However, conventional DMARDs such as Methotrexate, gold compounds, antimalarials, cyclosporine A, leflunomide, azathioprine, sulfasalazine d-penicillamine, aurothioglucose or auranofin) are all associated with toxicity, and although Methotrexate remains the standard of care in the United States and Europe for patients with RA, a response of less than 50%. improvement is often observed.

Methotrexate is considered to be the gold standard treatment for patients who have moderate to severe rheumatoid arthritis with pain, stiffness, swelling, and fatigue. Methotrexate is an antimetabolite which interferes with the way cells utilize essential nutrients. As a result, methotrexate inhibits the activity of the immune system, consequently reducing inflammation. As a cytotoxic drug it may slow the rapid growth of cells in the synovial membrane that lines the joints. However, Methotrexate is a potent drug in which many fear its potential toxicity and question the safety of long-term use of the drug.

As such, there is interest in the development of additional methods and compositions for treating RA in which the therapeutic efficacy of known compositions are improved. In addition, combination treatment may be employed to decrease the doses of the individual components in the resulting combinations while still preventing unwanted or harmful side effects of the individual components. Thus, there is an urgent need to discover suitable methods for the treatment of RA, including monotherapy and combination treatments that result in reduction of toxicity, decreased side effects and effective treatment.

### SUMMARY OF THE INVENTION

The present invention provides methods of treating rheumatoid arthritis. The methods generally involve an intermittent dosing strategy for administering Atiprimod alone or in combination with a second therapeutic agent wherein the second therapeutic agent is selected from the group consisting of Methotrexate, gold compounds, antimalarials, cyclosporine A, leflunomide, azathioprine, sulfasalazine d-penicillamine, aurothioglucose, auranofin or TNF inhibitors.

One embodiment of the present invention provides a method of treating rheumatoid arthritis comprising administering to a mammal a therapeutically effective amount of Atiprimod (the compound represented by the following Formula (I), or pharmaceutically acceptable salt, hydrate, or solvate thereof) according to the subject intermittent dosing strategy:

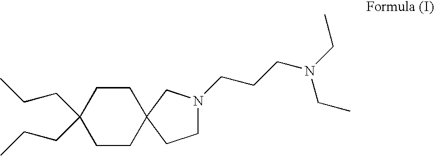

For example, the method of the invention comprises administration of a dimaleate salt of the compound of Formula I.

### BRIEF DESCRIPTION OF THE DRAWINGS

FIG. 1 depicts the Formula of Atiprimod (2-(3-Diethylaminopropyl)-8,8-dipropl-2-azaspiro[4,5]decane dimaleate.

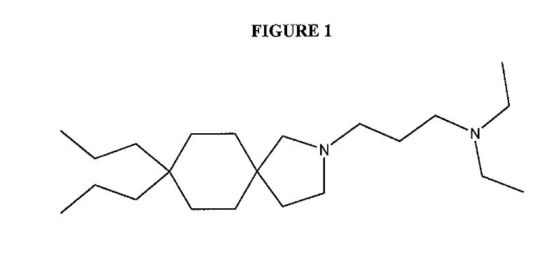

FIG. 2 provides Atiprimod PK modeling data in which 120 mg is given once daily on Days 1, 2, and 3, and then 40 mg is given once weekly starting on Day 8.

### DEFINITIONS

As used herein, “RA” refers to rheumatoid arthritis which is an autoimmune disease that causes chronic inflammation of the joints, the tissue around the joints, as well as other organs in the body

“Treatment” or “treating' as used herein means any therapeutic intervention in a subject, usually a mammalian subject, generally a human subject, including: (i) prevention, that is, causing the clinical symptoms not to develop, e.g., preventing progression to a harmful state; (ii) inhibition, that is, arresting the development or further development of rheumatoid arthritis.

As used herein, “subject' or “individual” or “patient” refers to any subject for whom or which therapy is desired, and generally refers to the recipient of the therapy to be practiced according to the invention. The subject can be any vertebrate, but will typically be a mammal. If a mammal, the subject will in many embodiments be a human, but may also be a domestic livestock, laboratory subject or pet animal.

“Compound” refers to the compound or salt, hydrate, or solvate thereof For example, the usage of the term Compound as in “a Compound represented by Formula 1” will be understood to mean “a compound represented by Formula 1 or pharmaceutically acceptable salt, hydrate, or solvate thereof”.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “symptom” includes a plurality of such symptoms associated with rheumatoid arthritis and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides methods of treating rheumatoid arthritis. The methods generally involve an intermittent dosing strategy for administering Atiprimod alone or in combination with at least a second therapeutic agent to an individual suffering from rheumatoid arthritis. In certain embodiment, the second therapeutic agent is selected from the group consisting of Methotrexate, gold compounds, antimalarials, cyclosporine A, leflunomide, azathioprine, sulfasalazine d-penicillamine, aurothioglucose, auranofin or TNF inhibitors.

The compounds useful in the methods of the present invention comprises Atiprimod, represented by the following Formula (I), or a salt, hydrate, or solvate thereof:

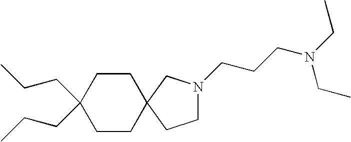

In some embodiments, a subject method of treating rheumatoid arthritis comprises administering Atiprimod and at least a second therapeutic agent. In certain embodiment, the second therapeutic agent is selected from the group consisting of Methotrexate, gold compounds, antimalarials, cyclosporine A, leflunomide, azathioprine, sulfasalazine d-penicillamine, aurothioglucose auranofin or TNF inhibitors.

### Intermittent Doasage Strategy

The subject methods include an intermittent dosing strategy for administering Atiprimod alone or in combination with at least a second therapeutic agent to an individual suffering from rheumatoid arthritis. In certain embodiment, the second therapeutic agent is selected from the group consisting of Methotrexate, gold compounds, antimalarials, cyclosporine A, leflunomide, azathioprine, sulfasalazine d-penicillamine, aurothioglucose, auranofin or or TNF inhibitors.

Aspects of the invention provide an intermittent Atiprimod dosing strategy that takes advantage of the drug's distinctive pharmacokinetic and distributive properties. The subject intermittent dosing strategy is based on the very long terminal half-life of Atiprimod. Experience from early atiprimod studies reports patients arthritis patients feeling better after single doses when higher amounts (with respect to RA treatment) of Atiprimod are given (e. g., after a single dosage of 90 mg). Also, Oral Atiprimod has a half-life of (60 to 67 days). The maximum concentration (Cₘₐₓ) and area under the concentration-time curve (AUC) after 14 or 28 days of daily oral dosing are lower than expected as a result of Atiprimod's long half-life. As such, the subject intermittent dosing strategy provides for an initial dose, which is below the MTD for Atiprimod, which utilizes advantageously the drug's extremely long half-life, while also allowing for benefits from rapid onset of relief that is associated with higher dosages of the drug (e. g., single doses of 90 mg).

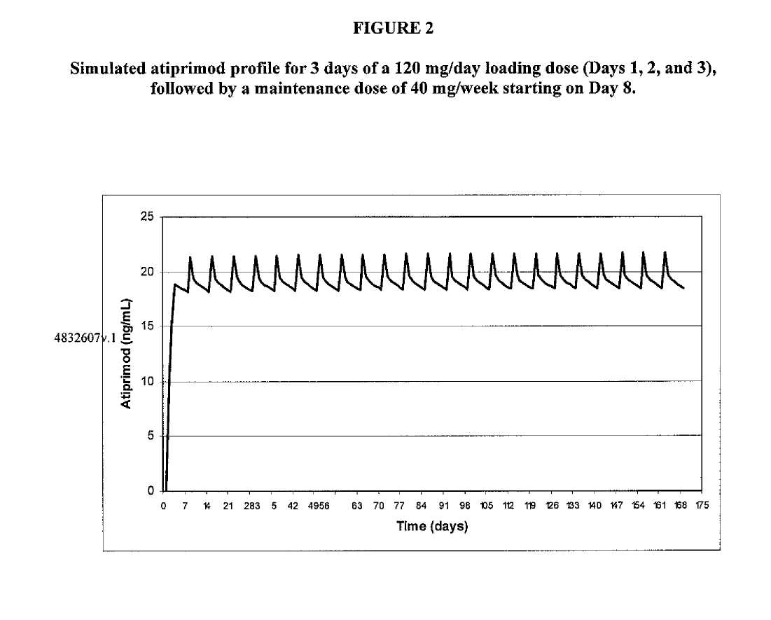

In certain aspects, the patient will receive a loading dose for 3, 4 or 5 days which is followed by a treatment free period the remainder of the first week. This will then be followed by weekly dosing, staring the first day of the second week. This weekly treatment may then proceed indefinitely on a chronic, once-weekly basis. FIG. 2 describes one embodiment of this approach, where treatment starts with three daily doses of 120 mg (allowing for rapid onset of therapeutic action), followed by a treatment-free period of four days, then commencing with weekly dosing of 40 mg, resulting in a near-steady-state blood level of 20 ng/dl.

Although the dosage used will vary depending on the clinical goals to be achieved, a suitable dose range is one which provides up to about 10 mg to about 500 mg of Atiprimod in a single daily dosage which is administered to a patient over a specific period of time. For example, about 10 mg, 30 mg, 60 mg, 90 mg, 120 mg, 150 mg, 180 mg, 210 mg, 240 mg, 270 mg, 300 mg, 330 mg, 360 mg, 390 mg, 420 mg, 450 mg, 480 mg, 500 mg and the like is administered to a patient as a single daily dosage, on a daily basis, a weekly basis or some other basis. Further, the patient may receive the specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

In the subject methods, the intermittent dosing strategy includes administering a higher daily dosage of Atiprimod to a patient for a specific period of time and then decreasing the dosage to a maintenance level. In certain aspects, the patient may not receive any dosage of Atiprimod during a specific time period.

In certain embodiments, the intermittent dosing strategy comprises administering a starting dose of Atiprimod to a patient suffering from rheumatoid arthritis, which will be the highest dose, i.e., the top dose of Atiprimod to be administered to the patient. The starting top dose of the subject methods may range from 240 mg/day to a dose of 10 mg/day. For example, the patient may receive a starting top dose of 240 m/day, 180 mg/day, 150 mg/day, 120 mg/day, 90 mg/day, 60 mg/day, 30 mg/day, 20 mg/day or 10 mg/day.

For example, when treating a patient for rheumatoid arthritis, the patient would receive a dosage of 120 mg/day of Atiprimod for 5 days followed by 2 days off of Atiprimod therapy, (i.e., the patient does not receive any dosage of Atiprimod). Afterwards, the patient would receive a single dosage of 90 mg/week of Atiprimod on a short-term or chronic basis.

In another example, the intermittent dose schedule of treatment a patient for rheumatoid arthritis is 120 mg/day for 3 days, followed by 4 days off of Atiprimod therapy (i.e., the patient does not receive any dosage of Atiprimod) for a total of 4 days. Afterwards, the patient would receive a single dosage of 90 mg/week of Atiprimod on a short-term or chronic basis.

For example, the subject protocol is followed in patients who have inflammation of the joints, the tissue around the joints, as well as other organs in the body and who are suffering from at least one of the following symptoms: increased pain and stiffness in the morning, swollen joints, deformed and misshapen joints, intense fatigue, decreased energy, muscle aches, decreased appetite, weight loss, fever, depression, problems sleeping, anemia, bumps occurring under the skin (rheumatoid nodules), inflamed blood vessels, bleeding stomach ulcers, inflammation of the heart's sac (pericarditis), inflammation of the heart muscle (myocarditis), lung problems or eye problems.

The amount of the compound administered to the patient is less than an amount that would cause toxicity in the patient. In certain embodiments, the amount of the compound that is administered to the patient is less than the amount that causes a concentration of the compound in the patient's plasma to equal or exceed the toxic level of the compound. The optimal amount of the compound that should be administered to the patient in the practice of the present invention will depend on the individual as well as the severity of the individual's symptoms.

In some embodiments, an “effective amount” of Atiprimod is an amount that results in a reduction of at least one pathological parameter associated with rheumatoid arthritis. Thus, e.g., in some embodiments, an effective amount of Atiprimod is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual having rheumatoid arthritis and not treated with Atiprimod.

### Combinational Therapies

It is apparent to a person skilled in the art that any one or more of the specific dosages and dosage schedules of Atiprimod discussed above is also applicable to an additional agent, such as Methotrexate, gold compounds, antimalarials, cyclosporine A, leflunomide, azathioprine, sulfasalazine d-penicillamine, aurothioglucose, auranofin or a TNF inhibitor when the additional agent is used in the combination treatment. Although the dosage used will vary depending on the clinical goals to be achieved, a suitable dose range is one which provides a therapeutic dosage of a second therapeutic agent in a daily dosage which is administered to a patient over a specific period of time. For example, about 50 mg, 100 mg, 200 mg, 300 mg, 400 mg, 500 mg, 600 mg, 700 mg, 800 mg, 900 mg, 1000 mg and the like is administered to a patient as a daily dosage. In certain aspects, the patient may receive multiple doses of a second therapeutic agent within one day. For example, the patient may receive a dosage of Methotrexate once a day, twice a day, three times a day and the like. Further, the patient may receive the specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

In one embodiment, the patient may receive 300 mg of Methotrexate three times a day in combination with the subject Atiprimod intermittent dosing strategy.

Moreover, the specific dosage and dosage schedule of Atiprimod alone or in combination with an additional agent, e.g., Methotrexante, can further vary, and the optimal dose, dosing schedule, and route of administration can be determined based upon the individual and the severity of the individual's symptoms.

For example, when administered in separate formulations, Atiprimod and Methotrexate can be administered substantially simultaneously (e.g., within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more.

### Formulations

In general, the Atiprimod compositions are prepared in a pharmaceutically acceptable composition for delivery to a host. Pharmaceutically acceptable carriers preferred for use with the Atiprimod compositions of the invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising an Atiprimod composition according to the present invention may also be lyophilized using means well known in the art, for subsequent reconstitution and use according to the invention.

In general, the pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. Preservatives and other additives may also be present such as, for example, antimicrobials, antioxidants, chelating agents, and inert gases and the like.

Absorption promoters, detergents and chemical irritants (e.g., keratinolytic agents) can enhance transmission of the Atiprimod compositions into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

A colloidal dispersion system may be used for targeted delivery of the Atiprimod compositions to specific tissue. Colloidal dispersion systems include macromolecule complexes, nanocapsules, microspheres, beads, and lipid-based systems including oil-in-water emulsions, micelles, mixed micelles, and liposomes.

### Routes of Administration

The subject therapeutic Atiprimod composition is administered to an individual using any available method and route suitable for drug delivery, including in vivo and ex vivo methods, as well as systemic, mucosal, and localized routes of administration.

Conventional and pharmaceutically acceptable routes of administration include inhalational routes, intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the desired effect and ADME (absorption, distribution, metabolism and excretion) characteristics.

The subject Atiprimod compositions can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

The route of administration depends, in part, on the individual suffering from rheumatoid arthritis and the specific severity of symptoms. Such means include inhalation of aerosol suspensions or insufflation of the compositions of the invention. Nebulizer devices, metered dose inhalers, and the like suitable for delivery of Atiprimod compositions to the nasal mucosa, trachea and bronchioli are well-known in the art and will therefore not be described in detail here. For general review in regard to intranasal drug delivery, see, e.g., Chien, Novel Drug Delivery Systems, Ch. 5 (Marcel Dekker, 1992).

Parenteral routes of administration other than inhalation administration include, but are not necessarily limited to, topical, transdermal, subcutaneous, intramuscular, intraorbital, intraspinal, intrasternal, and intravenous routes, i.e., any route of administration other than through the alimentary canal. Parenteral administration can be carried to effect systemic or local delivery of the subject Atiprimod compositions.

Systemic administration typically involves intravenous, intradermal, subcutaneous, or intramuscular administration or systemically absorbed topical or mucosal administration of pharmaceutical preparations. Mucosal administration includes administration to the respiratory tissue, e.g., by inhalation, nasal drops, and the like.

The subject Atiprimod compositions can also be delivered to the subject by enteral administration. Enteral routes of administration include, but are not necessarily limited to, oral and rectal (e.g., using a suppository) delivery.

Methods of administration of a therapeutic Atiprimod composition through the skin or mucosa include, but are not necessarily limited to, topical application of a suitable pharmaceutical preparation, transdermal transmission, injection and epidermal administration. For transdermal transmission, absorption promoters or iontophoresis are suitable methods. For review regarding such methods, those of ordinary skill in the art may wish to consult Chien, supra at Ch. 7. Iontophoretic transmission may be accomplished using commercially available “patches” which deliver their product continuously via electric pulses through unbroken skin for periods of several days or more. An exemplary patch product for use in this method is the LECTRO PATCH™ (manufactured by General Medical Company, Los Angeles, Calif.) which electronically maintains reservoir electrodes at neutral pH and can be adapted to provide dosages of differing concentrations, to dose continuously and/or to dose periodically.

Epidermal administration can be accomplished by mechanically or chemically irritating the outermost layer of the epidermis sufficiently to provoke an immune response to the irritant. An exemplary device for use in epidermal administration employs a multiplicity of very narrow diameter, short tynes which can be used to scratch a therapeutic nucleic acid coated onto the tynes into the skin. The device included in the MONO-VACC™ tuberculin test (manufactured by Pasteur Merieux, Lyon, France) is suitable for use in epidermal administration of the subject Atiprimod compositions.

### Subjects Suitable for Treatment

Subjects suitable for treatment with a subject monotherapy or combination therapy treatment strategy include individuals suffering from rheumatoid arthritis. As such, the subject methods at least provide for improvement in relieving pain, reducing inflammation, slowing down or stopping joint damage, and improving the subject's sense of well-being and ability to function.

### Examples

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

125 patients with rheumatoid arthritis (RA) are distributed across five Groups in a 6-month, randomized, double-blinded, placebo-controlled, multi-center trial. All patients are given baseline assessments of disease via MRI imaging, standardized rheumatology scores, certain biomarkers (e. g., C-reactive peptide) and other measurements as appropriate. The same assessments are made at six months; some of these measurements are made at interim points.

All patients take single daily doses on Days 1, 2, 3, 4 and 5, and then single weekly doses starting Day 8.

Group 1 takes placebo doses for all doses.

Group 2 takes daily doses of 60 mg for Days 1, 2 and 3, followed by placebo doses on Days 4 and 5, then followed by weekly doses of 20 mg starting on Day 8.

Group 3 takes daily doses of 120 mg for Days 1, 2 and 3, followed by placebo doses on Days 4 and 5, then followed by weekly doses of 40 mg starting on Day 8.

Group 4 takes daily doses of 120 mg for Days 1, 2, 3 and 4, followed by placebo doses on Day 5, then followed by weekly doses of 60 mg starting on Day 8.

Group 5 takes daily doses of 120 mg for Days 1, 2, 3, 4 and 5, followed by weekly doses of 90 mg starting on Day 8.

The patients take doses for six months. An interim analysis of progress on signs and symptoms is made at three months without =blinding the study to investigators nor patients. At six months, assessments are made as to signs and symptoms as well as MRI imaging of disease modification. MRI evidence of disease modification among the treated groups vs. placebo will be the primary endpoint of the study.

What is claimed is:

1. A method for treating rheumatoid arthritis in an individual, the method comprising administering a non-toxic, effective amount of Atiprimod according to an intermittent dosage strategy.

2. The method according to claim 1, wherein the intermittent dosage strategy comprises:
administering an initial non-toxic daily dosage of Atiprimod to the individual for a specific period of time; and
following up with weekly dosages of atiprimod.

3. The method according to claim 2, wherein the initial Atiprimod dosage ranges from about 10 mg to about 500 mg in a single daily dosage and the maintenance dosages range from 10 mg to about 250 mg.

4. The method according to claim 2, wherein the initial dosage of Atiprimod starts at 120 mg/day for three days, followed by weekly doses starting on Day 8 of 40 mg/day.

5. The method according to claim 2, further comprising administering an effective amount of a second therapeutic agent selected from the group consisting of Methotrexate, gold compounds, antimalarials, cyclosporine A, leflunomide, azathioprine, sulfasalazine d-penicillamine, aurothioglucose, auranofin or a TNF inhibitor.

6. The method according to claim 5, wherein the second therapeutic agent is Methotrexate.

7. The method according to claim 6, wherein the Atiprimod and Methotrexate are administered orally.

8. An intermittent dosing strategy for treating an individual suffering from rheumatoid arthritis, the strategy comprising:
administering a starting top, daily dose of Atiprimod ranging from 10 mg/day to 120 mg/day for 3 to 5 days, followed by weekly dosages of 10 to 120 mg.

9. The method according to claim 8, wherein Atiprimod is administered in the form of a granule, tablet, pill, suppository, capsule, suspension, salve, or lotion.

10. The method according to claim 8, further comprising administering an effective amount of Methotrexate.

11. A method of treating a patient suffering from rhemuatoid arthritis comprising: Administering Atiprimod according to an intermittent dosing strategy comprising an initial high loading dose of Atiprimod followed by a series of incrementally lower de-escelating doses until a final maintenance dose is reached.

12. The intermittent dosing strategy of claim 11, wherein the first initial high loading dose is administered for 14 days followed by 14 days of no administration.

13. The intermittent dosing strategy of claim 12, wherein the lower de-escelating doses are correlated to Atiprimod's half-life.

14. The intermittent dosing strategy of claim 11, wherein the lower de-escelating doses are defined by a subject's liver function.
